(19) United States
(12) Patent Application Publication
(10) Pub. No.: US 2026/0157948 A1
(43) Pub. Date: Jun. 11, 2026

(54) PROCESS FOR DYEING AND/OR LIGHTENING KERATIN FIBERS

(71) Applicant: L'OREAL, Paris (FR)

(72) Inventors: Sarah BOULEMNAKHER, SAINT-OUEN (FR); Marie GIAFFERI, SAINT-OUEN (FR); Nicolas ALBISETTI, CLICHY (FR); Céline BOSSARD, SAINT-OUEN (FR); Stephan GLASSER, SAINT-OUEN (FR)

(21) Appl. No.: 18/703,978

(22) PCT Filed: Oct. 25, 2022
(86) PCT No.: PCT/EP2022/079806

§ 371 Date: Apr. 23, 2024

(30) Foreign Application Priority Data
Oct. 26, 2021 (FR) FR2111354

(51) Int. Cl. A61K 8/60; A61K 8/22; A61K 8/24; A61K 8/34; A61K 8/36; A61K 8/41; A61K 8/49; A61K 8/67; A61K 8/73; A61Q 5/10

(52) U.S. Cl. CPC A61K 8/604; A61K 8/22; A61K 8/24; A61K 8/342; A61K 8/345; A61K 8/361; A61K 8/41; A61K 8/411; A61K 8/49; A61K 8/492; A61K 8/4926; A61K 8/676; A61K 8/73; A61Q 5/10; A61K 2800/4324; A61K 2800/884

(57) ABSTRACT

The invention relates to a process for dyeing and/or lightening keratin fibers, comprising the following steps, in the order:
a) simultaneously or successively pouring a dyeing and/or lightening composition (A) comprising one or more alkaline agents and/or one or more dyeing agents and an oxidizing composition (B) comprising one or more chemical oxidants into a pot having a base with an inside area S;
b) mixing the two compositions (A) and (B) in the pot in order to obtain a composition (C) occupying a volume V in the pot, the ratio V/S being between 8 and 30 mm;
c) taking a sample of the composition (C) from the pot by hand;
d) applying the composition (C) to the keratin fibers by hand;
e) optionally, hand-massaging the keratin fibers;
f) after a leave-on time ranging from 1 min to 1 hour, rinsing the keratin fibers.

The invention relates to a process for dyeing and/or lightening keratin fibers, preferably the hair, wherein a dyeing and/or lightening composition is applied to the keratin fibers by hand.

Many people have sought for a long time to modify the color of their hair and in particular to mask their gray hair.

It is known practice to dye keratin fibers, in particular human keratin fibers such as the hair, to obtain “permanent” colorings with dyeing compositions containing oxidation dye precursors, notably oxidation bases, such as ortho- or para-phenylenediamines, ortho- or para-aminophenols, or heterocyclic compounds such as pyrazoles, pyrazolinones or pyrazolo-pyridines. These oxidation bases are colorless or weakly colored compounds which, when combined with oxidizing products, may give rise to colored compounds via a process of oxidative condensation.

It is also possible to vary the shades obtained with these oxidation bases by combining them with couplers or color modifiers. The variety of the molecules involved as oxidation bases and couplers makes it possible to obtain a rich palette of colors.

Permanent dyeing processes thus consist in using, with the dye composition containing the oxidation dyes, an aqueous composition comprising at least one oxidizing agent, such as hydrogen peroxide, under alkaline pH conditions in the vast majority of cases. The alkaline agent conventionally used is aqueous ammonia or can be chosen from other alkaline agents, such as alkanolamines.

The compositions containing the dyes and the oxidizing compositions are packaged separately and must be mixed just before application to the hair.

Generally, oxidation dye products intended for dyeing the hair at home are constituted of kits comprising, on the one hand, a dispenser bottle containing the oxidizing composition and, on the other hand, a tube containing the composition comprising the dyes. The bottle and the tube are packaged in a box which sometimes also contains a hair dye aftercare product and accessories such as gloves.

At the time of use, the user must empty the tube into the dispenser bottle in such a way as to mix the oxidizing composition with the dye composition. It is then necessary to shake the bottle in order to obtain a homogeneous mixture. The user can then dispense the ready-to-use composition on their hair by means of the dispenser bottle.

The application gesture is not simple and sometimes results in a dyeing result that is not uniform. The user often has the impression that they are not controlling the mixture.

Consumers are moreover in search of dyeing and/or lightening products that are more environmentally friendly, notably based on ingredients of natural origin, without compromising the dyeing and/or lightening properties obtained, and which limit the use of plastics.

Moreover, dyeing and/or lightening compositions often comprise starting materials for which the regulatory conditions are increasingly strict. It is therefore necessary to develop dyeing and/or lightening processes which use compositions comprising alternative compounds.

There thus exists a real need to provide a process for dyeing and/or lightening keratin fibers, in particular human keratin fibers such as the hair, which does not have the drawbacks mentioned above, and notably which is pleasant to use, which makes a mixing and application gesture easy, and which is more environmentally friendly, without compromising the final dyeing and/or lightening result.

These aims and others are achieved by the present invention, a subject of which is thus a process for dyeing and/or lightening keratin fibers, preferably the hair, comprising the following steps, in the order:
a) simultaneously or successively pouring a dyeing and/or lightening composition (A) comprising one or more alkaline agents and/or one or more dyeing agents and an oxidizing composition (B) comprising one or more chemical oxidants into a pot having a base with an inside area S;
b) mixing the two compositions (A) and (B) in the pot in order to obtain a composition (C) occupying a volume V in the pot, the ratio V/S being between 8 and 30 mm;
c) taking a sample of the composition (C) from the pot by hand;
d) applying the composition (C) to the keratin fibers by hand;
e) optionally, hand-massaging the keratin fibers;
f) after a leave-on time ranging from 1 min to 1 hour, preferably from 10 to 50 min, preferentially from 15 to 45 min, rinsing the keratin fibers.

According to one preferred embodiment, the process according to the invention is a process for dyeing keratin fibers, notably the hair.

For the purposes of the present invention, the expression taking a sample or applying “by hand” is intended to mean taking a sample or applying a composition directly with the hand, without an application utensil such as a fine brush or a brush, or else without a dispensing nozzle. The hand is preferably covered with a glove.

The process according to the invention allows an easy, pleasant application, as with the application of a hair care product, by virtue of a hand gesture. The application is also precise and localized.

Moreover, the mixing of the compositions is simple and safe. The configuration of the pot makes it possible notably to easily mix the compositions therein, limiting the risk of overflow while at the same time making it possible to easily access the bottom of the pot for taking by hand a sample of the composition to be applied.

The steps of mixing and taking a sample of the composition obtained are thus facilitated, thereby allowing a uniform application which results in good dyeing results.

In addition, the compositions used in the process according to the invention have good working qualities, notably a creamy, melting texture allowing quick and easy mixing, easy application by hand with limited risks of running, and easy and uniform spreading over the entire head of hair.

Moreover, when it is used to dye hair, the process according to the invention makes it possible to produce chromatic, powerful, intense and sparingly selective colorings, i.e. colorings that are uniform along the length of the fiber. It also makes it possible to obtain particularly good coverage of depigmented keratin fibers such as gray hair.

Furthermore, the pot has an internal volume which means that it can house all the constituents necessary for the dyeing and/or lightening process. The pot can thus both serve to separately contain the compositions (A) and (B) to be mixed and serve as a container for the mixing of these compositions, thus limiting waste.

Other subjects, features, aspects and advantages of the invention will emerge even more clearly on reading the description and the examples that follow.

In the text which follows, unless otherwise indicated, the limits of a range of values are included in that range, notably in the expressions “between” and “ranging from . . . to . . . ”.

Moreover, the expression “at least one” used in the present description is equivalent to the expression “one or more”.

The process for dyeing and/or lightening keratin fibers according to the invention uses a dyeing and/or lightening composition (A) comprising one or more alkaline agents and/or one or more dyeing agents and an oxidizing composition (B) comprising one or more chemical oxidants.

1. Dyeing and/or Lightening Composition (A)

The composition (A) comprises one or more alkaline agents and/or one or more dyeing agents.

The composition (A) preferably comprises at least one alkaline agent.

The composition (A) preferably comprises at least one dyeing agent.

The composition (A) preferably comprises at least one alkaline agent and at least one dyeing agent, the dyeing agent preferably being chosen from oxidation dyes.

### Alkaline Agent

The alkaline agents that are useful according to the invention may be mineral, organic or hybrid alkaline agent or agents.

For the purposes of the present invention, the terms “alkaline agent” and “basifying agent” are used interchangeably.

The mineral basifying agent(s) are preferably chosen from aqueous ammonia, alkali metal carbonates or bicarbonates such as sodium (hydrogen) carbonate and potassium (hydrogen) carbonate, alkali metal or alkaline-earth metal phosphates such as sodium phosphates or potassium phosphates, sodium or potassium hydroxides, alkali metal or alkaline-earth metal silicates or metasilicates such as sodium metasilicate, and mixtures thereof.

The organic basifying agent(s) are preferably chosen from alkanolamines, amino acids, organic amines other than alkanolamines and amino acids, oxyethylenated and/or oxypropylenated ethylenediamines, 1,3-diaminopropane, spermine or spermidine, and mixtures thereof.

The term “alkanolamine” means an organic amine comprising a primary, secondary or tertiary amine function, and one or more linear or branched C₁-C₈ alkyl groups bearing one or more hydroxyl radicals.

Organic amines chosen from alkanolamines such as monoalkanolamines, dialkanolamines or trialkanolamines comprising one to three identical or different C₁-C₄ hydroxyalkyl radicals are in particular suitable for performing the invention.

In particular, the alkanolamine(s) are chosen from monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl)aminomethane and mixtures thereof.

Advantageously, the amino acids are basic amino acids comprising an additional amine function. Such basic amino acids are preferably chosen from histidine, lysine, arginine, ornithine and citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole. The organic amine may also be chosen from amino acid dipeptides. As amino acid dipeptides that May be used in the present invention, mention may notably be made of carnosine, anserine and balenine. The organic amine may also be chosen from compounds including a guanidine function. As amines of this type other than arginine that may be used in the present invention, mention may notably be made of creatine, creatinine, 1,1-dimethylguanidine, 1,1-diethylguanidine, glycocyamine, metformin, agmatine, n-amidoalanine, 3-guanidinopropionic acid, 4-guanidinobutyric acid and 2-([amino(imino)methyl]amino) ethane-1-sulfonic acid.

Use may be made in particular of guanidine carbonate or monoethanolamine hydrochloride as hybrid compounds.

The alkaline agent(s) that are useful according to the invention is (are) preferably chosen from alkanolamines such as monoethanolamine, diethanolamine or triethanolamine; aqueous ammonia, carbonates or bicarbonates such as sodium (hydrogen) carbonate and potassium (hydrogen) carbonates, alkali metal or alkaline-earth metal silicates or metasilicates such as sodium metasilicate, basic amino acids, such as arginine, and mixtures thereof, more preferentially from alkanolamines and aqueous ammonia, better still from alkanolamines and even better still monoethanolamine.

In a particular embodiment, the composition according to the invention is free of aqueous ammonia.

The total content of the alkaline agent(s), when they are present, in the composition (A) ranges, preferably, from 0.1% to 40% by weight, more preferentially from 1% to 30% by weight, better still from 2% to 25% by weight, and even better still from 4% to 20% by weight, relative to the total weight of the composition (A).

According to one particular embodiment, the total content of alkanolamine, preferably of monoethanolamine, preferably ranges from 0.1% to 40% by weight, more preferentially from 1% to 30% by weight, better still from 2% to 25% by weight, even better still from 4% to 20% by weight, or even from 8% to 15% by weight, relative to the total weight of the composition (A).

According to one embodiment, the pH of the composition (A) according to the invention is between 8 and 13; preferably between 9 and 12.

The pH of the composition (A) may be adjusted to the desired value by means of acidic or alkaline agent(s) commonly used in the dyeing of keratin fibers, such as those described previously, or alternatively using buffer systems known to those skilled in the art.

### Dyeing Agents

The composition (A) according to the invention may comprise one or more dyeing agent(s).

Preferably, the composition (A) according to the invention comprises one or more dyes.

The dyes are chosen from direct dyes, oxidation dyes and mixtures thereof.

These direct dyes may be synthetic or natural.

The term “direct dye” is understood to mean colored entities. These are dyes that will spread superficially on the fiber.

These synthetic direct dyes are, for example, chosen from the dyes conventionally used for direct dyeing, and among which mention may be made of all the aromatic and/or non-aromatic dyes that are commonly used, such as nitrobenzene, azo, hydrazono, nitro(hetero)aryl, tri(hetero)arylmethane, (poly)methine, carbonyl, azine, porphyrin, metalloporphyrin, quinone and in particular anthraquinone, indoamine and phthalocyanine direct dyes, and mixtures thereof.

Among the nitrobenzene direct dyes, mention may be made of: 1,4-diamino-2-nitrobenzene, 1-amino-2-nitro-4-β-hydroxyethylaminobenzene, 1-amino-2-nitro-4-bis(β-1,4-bis(β-hydroxyethylamino)-2-nitrobenzene, 1-β-hydroxyethyl)aminobenzene, hydroxyethylamino-2-nitro-4-bis(β-hydroxyethylamino)benzene, 1-β-hydroxyethylamino-2-1-β-hydroxyethylamino-2-nitro-4-(ethyl) (β-nitro-4-aminobenzene, hydroxyethyl)aminobenzene, 1-amino-3-methyl-4-β-hydroxyethylamino-6-nitrobenzene, 1-1,2-diamino-4-nitrobenzene, 1-amino-2-nitro-4-β-hydroxyethylamino-5-chlorobenzene, amino-2-β-hydroxyethylamino-5-nitrobenzene, 1,2-bis(β-hydroxyethylamino)-4-nitrobenzene, 1-amino-2-tris(hydroxymethyl)methylamino-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-β-hydroxyethyloxy-2-β-hydroxyethylamino-5-nitrobenzene, 1-methoxy-2-β-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-β-hydroxyethylamino-4-β,γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-β-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-bis(β-hydroxyethyl)amino-3-nitrobenzene, 1-β-hydroxyethylamino-2-nitrobenzene, 1-hydroxy-4-β-hydroxyethylamino-3-nitrobenzene.

Among the azo direct dyes, mention may be made of: Basic Red 51, Basic Orange 31, Disperse Red 17, Acid Yellow 9, Acid Black 1, Basic Red 22, Basic Red 76, Basic Yellow 57, Acid Yellow 36, Acid Orange 7, Acid Red 33, Acid Red 35, Acid Yellow 23, Acid Orange 24, Disperse Black 9, Basic Brown 16, Basic Brown 17.

Among the hydrazono direct dyes, mention may be made of: Basic Yellow 87.

Among the nitroaryl direct dyes, mention may be made of: HC Blue 2, HC Yellow 2, HC Red 3,4-hydroxypropylamino-3-nitrophenol, N,N′-bis(2-hydroxyethyl)-2-nitrophenylenediamine.

Among the triarylmethane direct dyes, mention may be made of: Basic Violet 1, Basic Violet 2, Basic Violet 3, Basic Violet 4, Basic Violet 14, Basic Blue 1, Basic Blue 7, Basic Blue 26, Basic Green 1, Basic Blue 77 (also known as HC Blue 15), Acid Blue 1; Acid Blue 3; Acid Blue 7, Acid Blue 9; Acid Violet 49; Acid Green 3; Acid Green 5; Acid Green 50.

Among the quinone direct dyes, mention may be made of: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and also the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4-bis(β,γ-dihydroxypropylamino) anthraquinone, Acid Blue 25, Acid Blue 43, Acid Blue 78, Acid Blue 129, Acid Blue 138, Acid Blue 140, Acid Blue 251, Acid Green 25, Acid Green 41, Acid Violet 42, Mordant Red 3, Acid Black 48, HC Blue 16.

Among the azine direct dyes, mention may be made of: Basic Blue 17, Basic Red 2.

Among the indoamine direct dyes, mention may be made of: 2-β-hydroxyethlyamino-5-[bis(β-4′-hydroxyethyl)amino]anilino-1,4-benzoquinone, 2-β-hydroxyethylamino-5-(2′-methoxy-4′-amino) anilino-1,4-benzoquinone, 3-N-(2′-chloro-4′-hydroxy)phenylacetylamino-6-methoxy-1,4-benzoquinoneimine, 3-N-(3′-chloro-4′-methylamino)phenylureido-6-methyl-1,4-benzoquinoneimine, 3-[4′-N-(ethylcarbamylmethyl)amino]phenylureido-6-methyl-1,4-benzoquinoneimine.

The natural direct dyes are chosen, for example, from lawsone, juglone, indigo, leuco indigo, indirubin, isatin, hennotannic acid, alizarin, carthamine, morin, purpurin, carminic acid, kermesic acid, laccaic acid, purpurogallin, protocatechaldehyde, curcumin, spinulosin, apigenidin, orceins, carotenoids, betanin, chlorophylls, chlorophyllines, monascus, polyphenols or ortho-diphenols.

Among the ortho-diphenols that are useful according to the invention, mention may be made of: catechin, quercetin, brazilin, haematein, haematoxylin, chlorogenic acid, caffeic acid, gallic acid, L-DOPA, cyanidin, (-)-epicatechin, (-)-epigallocatechin, (-)-epigallocatechin 3-gallate (EGCG), isoquercetin, pomiferin, esculetin, 6,7-dihydroxy-3-(3-hydroxy-2,4-dimethoxyphenyl) coumarin, santalin A and B, mangiferin, butein, maritimetin, sulfuretin, robtein, betanidin, pericampylinone A, theaflavin, proanthocyanidin A2, proanthocyanidin B2, proanthocyanidin C1, procyanidins DP 4-8, tannic acid, purpurogallin, 5,6-dihydroxy-2-methyl-1,4-naphthoquinone, alizarin, wedelolactone and natural extracts containing same.

When the composition (A) comprises at least one direct dye, they are preferably present in a total content ranging from 0.001% to 20% by weight, preferably from 0.005% to 15% by weight, more preferentially from 0.01% to 10% by weight, better still from 0.05% to 5%, and even better still from 0.1% to 3% by weight, relative to the weight of the composition.

Preferably, the composition (A) according to the invention comprises one or more dyes chosen from oxidation dyes.

The oxidation dyes may be chosen from one or more oxidation bases, optionally in combination with one or more couplers.

Preferably, the oxidation dye(s) comprise one or more oxidation bases.

Preferably, the composition according to the invention comprises one or more oxidation bases.

The oxidation bases may be present in the form of salts, solvates and/or solvates of salts.

The addition salts of the oxidation bases present in the composition according to the invention are notably chosen from the addition salts with an acid, such as the hydrochlorides, hydrobromides, sulfates, citrates, succinates, tartrates, lactates, tosylates, benzenesulfonates, methanesulfonates, phosphates and acetates, and the addition salts with a base such as sodium hydroxide, potassium hydroxide, aqueous ammonia, amines or alkanolamines.

Moreover, the solvates of the oxidation bases more particularly represent the hydrates of said oxidation bases and/or the combination of said oxidation bases with a linear or branched C₁ to C₄ alcohol such as methanol, ethanol, isopropanol or n-propanol. Preferably, the solvates are hydrates.

By way of example, the oxidation bases are chosen from para-phenylenediamines, bis(phenyl)alkylenediamines, para-aminophenols, ortho-aminophenols, heterocyclic bases and the corresponding addition salts, solvates and/or solvates of the salts.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-(γ-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl) pyrrolidine, and the addition salts, solvates and/or solvates of salts thereof.

Among the para-phenylenediamines mentioned above, particular preference is given to para-phenylenediamine, para-toluenediamine, 2-isopropyl-para-phenylenediamine, 2-β-hydroxyethyl-para-phenylenediamine, 2-(γ-hydroxypropyl)-para-phenylenediamine, 2-methoxymethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts, solvates and/or solvates of salts thereof.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts, the solvates and/or the solvates of the salts.

Among the para-aminophenols that are mentioned are, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl) phenol and 4-amino-2-fluorophenol, and the addition salts, the solvates and/or the solvates of the salts.

Among the ortho-aminophenols that may be mentioned, for example, are 2-aminophenol, 2-amino-5-methylphenol, 2-amino-6-methylphenol and 5-acetamido-2-aminophenol, and the corresponding addition salts, the solvates and the solvates of the salts.

Among the heterocyclic bases that may be mentioned, for example, are pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts, the solvates and the solvates of the salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl) ethanol, 2-(3-aminopyrazolo[1,5-a]pyrid-7-yl) ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine, and the corresponding addition salts, the solvates and the solvates of the salts.

More particularly, the oxidation bases that are useful in the present invention are chosen from 3-aminopyrazolo[1,5-a]pyridines and preferably substituted on carbon atom 2 with:
a) a (di)(C1-C6) (alkyl)amino group, said alkyl group possibly being substituted with at least one hydroxyl, amino or imidazolium group;
b) an optionally cationic 5- to 7-membered heterocycloalkyl group containing from 1 to 3 heteroatoms, optionally substituted with one or more (C1-C6)alkyl groups, such as a di(C1-C4)alkylpiperazinium group; or
c) a (C1-C6)alkoxy group optionally substituted with one or more hydroxyl groups, such as a β-hydroxyalkoxy group, and the corresponding addition salts, the solvates and the solvates of the salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof, the solvates and the solvates of the salts thereof, and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the corresponding addition salts, the solvates and/or the solvates of the salts. Use may also be made of 4,5-diamino-1-(β-methoxyethyl)pyrazole.

A 4,5-diaminopyrazole will preferably be used and even more preferentially 4,5-diamino-1-(β-hydroxyethyl)pyrazole and/or a corresponding salt, a solvate and/or a solvate of a salt.

The pyrazole derivatives that may also be mentioned comprise diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one, the salts thereof, the solvates thereof and the solvates of the salts thereof.

Use will preferably be made of 2,3-diamino-6,7-dihydro-1H,5H-pyrazolo[1,2-a]pyrazol-1-one and/or a corresponding salt, solvate and/or solvate of a salt.

Use will preferably be made, as heterocyclic bases, of 4,5-diamino-1-(β-hydroxyethyl)pyrazole and/or 2,3-diamino-6,7-dihydro-1H, 5H-pyrazolo[1,2-a]pyrazol-1-one and/or 2-β-hydroxyethoxy-3-aminopyrazolo[1,5-a]pyridine and/or a corresponding salt, solvate and/or solvate of a salt.

Preferably, the oxidation base(s) is (are) chosen from para-phenylenediamines, bis(phenyl)alkylenediamines, para-aminophenols, ortho-aminophenols, heterocyclic bases, and the corresponding addition salts, the solvates thereof and/or the solvates of the salts thereof, and mixtures thereof; more preferentially from 2-methoxymethyl-para-phenylenediamine, 2-β-hydroxyethyl-para-phenylenediamine, 2-γ-hydroxypropyl-para-phenylenediamine, and the addition salts thereof, the solvates thereof and/or the solvates of the salts thereof, and mixtures thereof.

When the composition (A) comprises at least one oxidation base, the oxidation base(s) is (are) preferably present in a total content ranging from 0.001% to 20% by weight, preferably from 0.005% to 15% by weight, more preferentially from 0.01% to 10% by weight, better still from 0.05% to 5%, even better still from 0.1% to 3% by weight, relative to the weight of the composition (A).

According to one preferred embodiment, when the composition (A) comprises at least one oxidation base chosen from 2-methoxymethyl-para-phenylenediamine, 2-β-hydroxyethyl-para-phenylenediamine, 2-γ-hydroxypropyl-para-phenylenediamine, the addition salts thereof, the solvates thereof and/or the solvates of the salts thereof and mixtures thereof, said base(s) is (are) present in a total content ranging from 0.001% to 20% by weight, preferably from 0.005% to 15% by weight, more preferentially from 0.01% to 10% by weight, better still from 0.05% to 5%, even better still from 0.1% to 3% by weight, relative to the total weight of the composition (A).

In one particular embodiment, the composition (A) according to the invention is free of oxidation bases chosen from para-phenylenediamine, para-toluenediamine, the addition salts thereof, the solvates thereof and the solvates of the salts thereof.

The oxidation dye(s) may also be chosen from one or more couplers, which may be chosen from the couplers conventionally used for the dyeing of keratin fibers.

Preferably, the composition (A) according to the invention comprises one or more couplers.

Among the couplers that are useful according to the invention, mention may be made in particular of meta-phenylenediamines, meta-aminophenols, meta-diphenols, naphthalene-based coupling agents and heterocyclic coupling agents, and also the corresponding addition salts, the solvates and solvates of the salts thereof.

Mention may be made, for example, of 6-hydroxybenzomorpholine, hydroxyethyl-3-4-methylenedioxyaniline, 2-amino-5-ethylphenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy) propane, 3-ureidoaniline, 3-ureido-1-dimethylaminobenzene, sesamol, α-naphthol, 2-methyl-1-naphthol, 6-hydroxyindole, 4-hydroxyindole, 4-hydroxy-N-methylindole, 2-amino-3-hydroxypyridine, 3,5-diamino-2,6-dimethoxypyridine, 2,6-bis(β-hydroxyethylamino) toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole and 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts, the solvates and the solvates of the salts thereof, and the corresponding mixtures.

Preferably, the coupler(s) are chosen from: 6-hydroxybenzomorpholine, the addition salts thereof, the solvates thereof and/or the solvates of the salts thereof, hydroxyethyl-3,4-methylenedioxyaniline, the addition salts thereof, the solvates thereof and/or the solvates of the salts thereof, 2-amino-5-ethylphenol, the addition salts thereof, the solvates thereof and/or the solvates of the salts thereof, and mixtures thereof.

In general, the addition salts of the couplers that may be used in the context of the invention are chosen in particular from addition salts with an acid, such as hydrochlorides, hydrobromides, sulfates, citrates, succinates, tartrates, lactates, tosylates, benzenesulfonates, phosphates and acetates, and the addition salts with a base such as sodium hydroxide, potassium hydroxide, aqueous ammonia, amines or alkanolamines.

Moreover, the solvates more particularly represent the hydrates of these couplers and/or the combination of these couplers with a linear or branched C1 to C4 alcohol such as methanol, ethanol, isopropanol or n-propanol. Preferably, the solvates are hydrates.

In one particular embodiment, the composition according to the invention is free of oxidation couplers chosen from resorcinol, 2-methylresorcinol, 4-chlororesorcinol, the addition salts thereof, the solvates thereof and the solvates of the salts thereof.

When the composition (A) comprises one or more oxidation couplers, the total content of the coupler(s) present in the composition according to the invention ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

When they are present, the total content of the couplers chosen from 6-hydroxybenzomorpholine, hydroxyethyl-3,4-methylenedioxyaniline, 2-amino-5-ethylphenol and also the addition salts thereof, the solvates thereof and/or the solvates of the salts thereof preferably ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

When they are present, the total content of the couplers chosen from 6-hydroxybenzomorpholine, the addition salts thereof, the solvates thereof and/or the solvates of the salts thereof preferably ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

When they are present, the total content of the couplers chosen from hydroxyethyl-3,4-methylenedioxyaniline, the addition salts thereof, the solvates thereof and/or the solvates of the salts thereof preferably ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

When they are present, the total content of the couplers chosen from 2-amino-5-ethylphenol, the addition salts thereof, the solvates thereof and/or the solvates of the salts thereof preferably ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

Preferably, the total content of the dyes ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

When they are present, the total content of the oxidation dyes preferably ranges from 0.001% to 20% by weight, more preferentially from 0.005% to 15% by weight, better still from 0.01% to 10% by weight, even better still from 0.05% to 5% by weight, and even better still from 0.1% to 3% by weight, relative to the total weight of the composition (A).

Alkyl (poly)glycoside

The composition (A) may also comprise one or more alkyl (poly)glycoside(s).

Preferably, the composition (A) comprises one or more alkyl (poly)glycoside(s).

The term “alkyl (poly)glycoside” denotes an alkyl polyglycoside or an alkyl monoglycoside, also referred to in the present patent application as an alkyl glycoside, which may be alkoxylated with one or more alkylene oxide groups, preferentially C₂-C₄ alkylene oxide groups.

The alkyl (poly)glycoside(s) may be represented by the general formula below: R₁O—(R₂O)ₜ-(G)ᵥ wherein:
R₁ represents a linear or branched alkyl or alkenyl radical including 6 to 24 carbon atoms and notably 8 to 18 carbon atoms, or an alkylphenyl radical of which the linear or branched alkyl radical includes 6 to 24 carbon atoms and notably 8 to 18 carbon atoms;
R₂ represents an alkylene radical including 2 to 4 carbon atoms,
G represents a sugar unit comprising 5 to 6 carbon atoms,
t denotes a value ranging from 0 to 10 and preferably from 0 to 4,
v denotes a value ranging from 1 to 15 and preferably from 1 to 4.

Preferably, the alkyl (poly)glycosides are compounds of the formula described above wherein:
R₁ denotes a linear or branched, saturated or unsaturated alkyl radical including from 8 to 18 carbon atoms,
R₂ represents an alkylene radical including 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl (poly)glycoside is an alkyl (poly) glucoside.

Among the commercial products, mention may be made of the products sold by COGNIS under the names Plantaren® (600 CS/U, 1200 and 2000) or Plantacare® (818, 1200 and 2000); the products sold by SEPPIC under the names Oramix CG 110 and Oramix® NS 10; the products sold by BASF under the name Lutensol GD 70; the products sold by CHEM Y under the name AG10 LK; the products sold by Evonik Goldschmidt under the trade names Tego Care CG 90 or Tego Care CG 90 MB, the products sold by SEPPIC under the trade names Montanov® 68, Montanov® 68 MB, Montanov® 14 or Montanov® 202, or the products sold by BASF under the name Emulgade® PL 68/50.

Preferentially, the composition comprises one or more alkyl (poly)glycosides chosen from the following, alone or as a mixture: (C6-C24 alkyl) (poly)glycosides, and more particularly (C8-C18 alkyl) (poly)glycosides, preferably chosen from coco glucoside, lauryl glucoside, caprylyl/capryl glucoside and cetearyl glucoside.

C8/C18 alkyl (poly) glucosides of 1-4 type, and notably the compounds having the INCI names coco glucoside and cetearyl glucoside, are most particularly preferred.

According to one preferred embodiment, the composition (A) comprises one or more alkyl (poly)glycosides chosen from the following, alone or as a mixture: (C14-C24 alkyl) (poly)glycosides, and more particularly (C14-C18 alkyl) (poly)glycosides, preferably chosen from cetearyl glucoside.

When it (they) is (are) present, the total content of alkyl (poly)glycoside(s) in the composition (A) preferably ranges from 0.01% to 20% by weight, preferentially from 0.02% to 10% by weight, more preferentially from 0.05% to 8% by weight, better still from 0.08% to 5% by weight, even better still from 0.1% to 3% by weight, or even from 0.2% to 2% by weight, relative to the total weight of the composition (A).

### Polysaccharide

The composition (A) may also comprise one or more polysaccharides, preferably chosen from anionic polysaccharides.

Preferably, the composition (A) comprises one or more polysaccharides, preferably chosen from anionic polysaccharides.

The term “polysaccharides” means polymers which contain at least 11 monosaccharide units. Preferentially, the polysaccharides of the invention include between 20 and 100 000 monosaccharide units.

The anionic polysaccharides according to the invention comprise one or more anionic or anionizable groups, and do not comprise any cationic or cationizable groups.

The anionic polysaccharides that are useful according to the invention may be chosen from those derived from the following sugars: glucose; galactose; arabinose; rhamnose; mannose; xylose; fucose; anhydrogalactose; galacturonic acid; glucuronic acid; mannuronic acid; galactose sulfate; anhydrogalactose sulfate.

The anionic polysaccharides of the invention may be natural or synthetic.

According to a particular embodiment, the anionic polysaccharides that are useful in the composition according to the invention are chosen from native gums such as:
tree or shrub exudates, for instance: acacia gum (branched polymer of galactose, arabinose, rhamnose and glucuronic acid); ghatti gum (polymer derived from arabinose, galactose, mannose, xylose and glucuronic acid); karaya gum (polymer derived from galacturonic acid, galactose, rhamnose and glucuronic acid); gum tragacanth (polymer of galacturonic acid, galactose, fucose, xylose and arabinose);
gums derived from algae, such as: alginates (polymers of mannuronic acid and glucuronic acid); carrageenans and furcellerans (polymers of galactose sulfate and anhydrogalactose sulfate);
microbial gums such as: xanthan gums (polymer of glucose, mannose acetate, mannose/pyruvic acid and glucuronic acid); gellan gums (polymer of partially acylated glucose, rhamnose and glucuronic acid).

For the purposes of the present invention, the term “microbial gums” means substances synthesized by fermentation of sugars by microorganisms.

According to one preferred embodiment, the anionic polysaccharides that are useful in the composition (A) are chosen from anionic gums, better still from anionic microbial gums, more preferentially from xanthan gums.

When they are present, the total content of polysaccharides preferably ranges from 0.01% to 10% by weight relative to the total weight of the composition (A), preferably from 0.05% to 8% by weight, better still from 0.1% to 5% by weight, relative to the total weight of the composition (A).

When they are present, the total content of anionic polysaccharides as defined previously preferably ranges from 0.01% to 10% by weight relative to the total weight of the composition (A), preferably from 0.05% to 8% by weight, better still from 0.1% to 5% by weight relative to the total weight of the composition (A).

The total content of the anionic microbial gums as defined previously ranges, when they are present, preferably from 0.01% to 10% by weight relative to the total weight of the composition (A), preferably from 0.05% to 8% by weight, better still from 0.1% to 5% by weight relative to the total weight of the composition (A).

Fatty Substances Other than Fatty Acids

The composition (A) may also comprise one or more fatty substance(s) other than fatty acids.

The term “fatty substance” means an organic compound that is insoluble in water at 25° C. and at atmospheric pressure (1.013×10⁵ Pa) (solubility of less than 5% by weight, preferably less than 1% by weight, even more preferentially less than 0.1% by weight). They bear in their structure at least one hydrocarbon chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The fatty substances that may be used in the present invention are neither (poly)oxyalkylenated nor (poly)glycerolated.

Preferably, the fatty substances that are useful according to the invention are non-silicone fatty substances.

The term “non-silicone fatty substance” refers to a fatty substance not containing any Si—O bonds and the term “silicone fatty substance” refers to a fatty substance containing at least one Si—O bond.

The fatty substances that are useful according to the invention may be liquid fatty substances (or oils) and/or solid fatty substances. The term “liquid fatty substance” means a fatty substance with a melting point of less than or equal to 25° C. at atmospheric pressure (1.013×10⁵ Pa) and the term “solid fatty substance” means a fatty substance with a melting point of greater than 25° C. at atmospheric pressure (1.013×10⁵ Pa).

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by TA Instruments. In the present patent application, all the melting points are determined at atmospheric pressure (1.013×10⁵ Pa).

More particularly, the liquid fatty substance(s) may be chosen from C6 to C16 liquid hydrocarbons, liquid hydrocarbons comprising more than 16 carbon atoms, non-silicone oils of animal origin, oils of triglyceride type of plant or synthetic origin, fluoro oils, liquid fatty alcohols, liquid esters of fatty acid and/or of fatty alcohol other than triglycerides, and mixtures thereof.

It is recalled that the fatty alcohols, esters and acids more particularly contain at least one saturated or unsaturated, linear or branched hydrocarbon group, comprising from 6 to 40, better still from 8 to 30 carbon atoms, which is optionally substituted, in particular, with one or more hydroxyl groups (in particular 1 to 4). If they are unsaturated, these compounds may comprise one to three conjugated or non-conjugated carbon-carbon double bonds.

As regards the C6 to C16 liquid hydrocarbons, these may be linear, branched, or optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly (or mineral oil), polydecenes, hydrogenated polyisobutene such as Parleam®, and mixtures thereof.

As hydrocarbon oils of animal origin, mention may be made of perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by Stearinerie Dubois or those sold under the names Miglyol® 810, 812 and 818 by Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by BNFL Fluorochemicals; perfluoro-1,2-dimethylcyclobutane; perfluoroalkanes such as dodecafluoropentane and tetradecafluorohexane, sold under the names PF 5050® and PF 5060® by 3M, or bromoperfluorooctyl sold under the name Foralkyl® by Atochem; nonafluoromethoxybutane and nonafluoroethoxyisobutane; perfluoromorpholine derivatives such as 4-trifluoromethylperfluoromorpholine sold under the name PF 5052® by 3M.

The liquid fatty alcohols that are suitable for use in the invention are more particularly chosen from linear or branched, saturated or unsaturated alcohols, preferably unsaturated or branched alcohols, comprising from 6 to 40 carbon atoms, preferably from 8 to 30 carbon atoms. These fatty alcohols are neither oxyalkylenated nor glycerolated. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof. Preferably, oleyl alcohol will be used.

As regards the liquid esters of fatty acids and/or of fatty alcohols, other than the triglycerides mentioned previously, mention may be made notably of esters of saturated or unsaturated, linear C1 to C26 or branched C3 to C26 aliphatic mono- or polyacids and of saturated or unsaturated, linear C1 to C26 or branched C3 to C26 aliphatic mono- or polyalcohols, the total carbon number of the esters being greater than or equal to 6 and more advantageously greater than or equal to 10.

Preferably, for the esters of monoalcohols, at least one from among the alcohol and the acid is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate, isopropyl palmitate, 2-ethylhexyl palmitate, 2-octyldecyl palmitate; alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Esters of C4 to C22 dicarboxylic or tricarboxylic acids and of C1 to C22 alcohols and esters of monocarboxylic, dicarboxylic or tricarboxylic acids and of C2 to C26 dihydroxy, trihydroxy, tetrahydroxy or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C6 to C30 and preferably C12 to C22 fatty acids. It is recalled that the term “sugar” refers to oxygen-bearing hydrocarbon compounds bearing several alcohol functions, with or without aldehyde or ketone functions, and which include at least 4 carbon atoms. These sugars may be monosaccharides, oligosaccharides or polysaccharides other than the anionic polysaccharides described hereinbelow.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described above and of linear or branched, saturated or unsaturated C6 to C30 and preferably C12 to C22 fatty acids. If they are unsaturated, these compounds may comprise one to three conjugated or non-conjugated carbon-carbon double bonds.

The esters may also be chosen from monoesters, diesters, triesters, tetraesters and polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmito-stearate esters.

More particularly, use is made of monoesters and diesters and notably sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by Amerchol, which is a methylglucose dioleate.

Preferably, use will be made of a liquid ester of a monoacid and of a monoalcohol.

According to one embodiment, the fatty substances, other than fatty acids, that are useful according to the invention are chosen from liquid fatty substances, preferably from liquid hydrocarbons containing more than 16 carbon atoms, plant oils, liquid fatty alcohols and liquid fatty esters, and mixtures thereof, more preferentially from liquid fatty alcohols.

Preferentially, the liquid fatty substance(s) are chosen from liquid fatty alcohols, in particular oleyl alcohol.

The solid fatty substances preferably have a viscosity of greater than 2 Pa·s, measured at 25° C. and at a shear rate of 1 s⁻¹.

The solid fatty substance(s) are preferably chosen from solid fatty alcohols, solid esters of fatty acids and/or of fatty alcohols, waxes, ceramides and mixtures thereof.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols may be saturated or unsaturated, and linear or branched, and include from 8 to 40 carbon atoms, preferably from 10 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 10 to 30, or even from 12 to 24 atoms and even better still from 14 to 22 carbon atoms.

The solid fatty alcohols that may be used are preferably chosen from saturated or unsaturated, linear or branched, preferably linear and saturated, (mono) alcohols including from 8 to 40 carbon atoms, better still from 10 to 30, or even from 12 to 24 and even better still from 14 to 22 carbon atoms.

The solid fatty alcohols that may be used may be chosen, alone or as a mixture, from: myristyl alcohol (or 1-tetradecanol); cetyl alcohol (or 1-hexadecanol); stearyl alcohol (or 1-octadecanol); arachidyl alcohol (or 1-eicosanol); behenyl alcohol (or 1-docosanol); lignoceryl alcohol (or 1-tetracosanol); ceryl alcohol (or 1-hexacosanol); montanyl alcohol (or 1-octacosanol); myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is chosen from cetyl alcohol, stearyl alcohol and mixtures thereof such as cetylstearyl alcohol or cetearyl alcohol.

The solid esters of a fatty acid and/or of a fatty alcohol that may be used are preferably chosen from esters derived from a C9-C26 carboxylic fatty acid and/or from a C9-C26 fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol, including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C4-C22 dicarboxylic or tricarboxylic acids and of C1-C22 alcohols and esters of monocarboxylic, dicarboxylic or tricarboxylic acids and of C2-C26 dihydroxy, trihydroxy, tetrahydroxy or pentahydroxy alcohols may also be used.

Mention may notably be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C9-C26 alkyl palmitates, notably myristyl palmitate, cetyl palmitate or stearyl palmitate; C9-C26 alkyl myristates, such as cetyl myristate, stearyl myristate and myristyl myristate; and C9-C26 alkyl stearates, notably myristyl stearate, cetyl stearate and stearyl stearate; and mixtures thereof.

For the purposes of the present invention, a wax is a lipophilic compound, which is solid at 25° C. and atmospheric pressure, with a reversible solid/liquid change of state, having a melting point greater than about 40° C. and which may range up to 200° C., and having, in the solid state, an anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to ambient temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may be made notably of hydrocarbon waxes, for instance beeswax, notably of organic origin, lanolin wax and Chinese insect waxes; rice bran wax, carnauba wax, candelilla wax, ouricury wax, esparto grass wax, berry wax, shellac wax, Japan wax and sumac wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of C20 to C60 microcrystalline waxes, such as Microwax HW. Mention may also be made of the MW 500 polyethylene wax sold under the reference Permalen 50-L polyethylene.

Mention may also be made of the waxes obtained by catalytic hydrogenation of animal or plant oils containing linear or branched C8 to C32 fatty chains. Among these waxes mention may notably be made of isomerized jojoba oil such as the trans-isomerized partially hydrogenated jojoba oil, notably the product produced or sold by Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane) tetrastearate, notably the product sold under the name Hest 2T-4S® by Heterene.

The waxes obtained by hydrogenation of castor oil esterified with cetyl alcohol, such as those sold under the names Phytowax Castor 16L64® and 22L73® by Sophim, may also be used.

A wax that may also be used is a C20-C40 alkyl (hydroxystearyloxy)stearate (the alkyl group containing from 20 to 40 carbon atoms), alone or as a mixture. Such a wax is notably sold under the names Kester Wax K 82 PR, Hydroxypolyester K 82 P® and Kester Wax K 80 P® by Koster Keunen.

It is also possible to use microwaxes in the compositions of the invention; mention may notably be made of carnauba microwaxes, such as the product sold under the name MicroCare 350® by Micro Powders, synthetic-wax microwaxes, such as the product sold under the name MicroEase 114SR by Micro Powders, microwaxes constituted of a mixture of carnauba wax and polyethylene wax, such as the products sold under the names Micro Care 300® and 310® by Micro Powders, microwaxes constituted of a mixture of carnauba wax and of synthetic wax, such as the product sold under the name Micro Care 325® by Micro Powders, polyethylene microwaxes, such as the products sold under the names Micropoly 200®, 220®, 220L® and 250SR by Micro Powders, and polytetrafluoroethylene microwaxes, such as the products sold under the names Microslip 519® and 519 L® by Micro Powders.

The waxes are preferably chosen from mineral waxes, for instance paraffin, petroleum jelly, lignite or ozokerite wax; plant waxes, for instance cocoa butter or cork fiber or sugar cane waxes, olive tree wax, rice wax, hydrogenated jojoba wax, ouricury wax, carnauba wax, candelilla wax, esparto grass wax, or absolute waxes of flowers, such as the essential wax of blackcurrant blossom sold by Bertin (France); waxes of animal origin, for instance beeswaxes or modified beeswaxes (cera bellina), spermaceti, lanolin wax and lanolin derivatives; microcrystalline waxes; and mixtures thereof.

The ceramides, or ceramide analogs such as glycoceramides, that may be used in the compositions according to the invention, are known; mention may be made in particular of ceramides of classes I, II, III and V according to the Dawning classification.

The ceramides or analogues thereof that may be used preferably correspond to the following formula: R³CH(OH)CH(CH₂OR²)(NHCOR¹), wherein:
R¹ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R² denotes a hydrogen atom, a (glycosyl) n group, a (galactosyl) m group or a sulfogalactosyl group, wherein n is an integer ranging from 1 to 4 and m is an integer ranging from 1 to 8;
R³ denotes a C₁₅-C₂₆ hydrocarbon group, saturated or unsaturated in the alpha position, this group possibly being substituted with one or more C₁-C₁₄ alkyl groups; it being understood that in the case of natural ceramides or glycoceramides, R³ may also denote a C₁₅-C₂₆ alpha-hydroxyalkyl group, the hydroxyl group being optionally esterified with a C₁₆-C₃₀ alpha-hydroxy acid.

The ceramides that are more particularly preferred are the compounds for which R¹ denotes a saturated or unsaturated alkyl derived from C₁₆-C₂₂ fatty acids; R² denotes a hydrogen atom and R₃ denotes a saturated linear C₁₅ group.

Preferentially, use is made of ceramides for which R¹ denotes a saturated or unsaturated alkyl group derived from C₁₄-C₃₀ fatty acids; R² denotes a galactosyl or sulfogalactosyl group; and R³ denotes a —CH═CH—(CH₂)₁₂—CH₃ group.

Use may also be made of compounds for which R¹ denotes a saturated or unsaturated alkyl radical derived from C₁₂-C₂₂ fatty acids; R² denotes a galactosyl or sulfogalactosyl radical and R³ denotes a saturated or unsaturated C₁₂-C₂₂ hydrocarbon radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-Oleoyldihydrosphingosine will preferably be used.

The solid fatty substances are preferably chosen from solid fatty alcohols, in particular from cetyl alcohol, stearyl alcohol and mixtures thereof such as cetylstearyl or cetearyl alcohol.

Butters may also be used.

For the purposes of the present invention, the term “butter” (also referred to as a “pasty fatty substance”) means a lipophilic fatty compound with a reversible solid/liquid change of state, comprising at a temperature of 25° C. and at atmospheric pressure (760 mmHg) a liquid fraction and a solid fraction. Preferably, the butter(s) according to the invention have a melting start temperature above 25° C. and a melting end temperature below 60° C.

Preferably, the particular butter(s) are of plant origin, such as those described in Ullmann's Encyclopedia of Industrial Chemistry (“Fats and Fatty Oils”, A. Thomas, published online: Jun. 15, 2000, DOI: 10.1002/14356007.a10_173, point 13.2.2.2. Shea Butter, Borneo Tallow, and Related Fats (Vegetable Butters)).

Mention may be made more particularly of shea butter, Nilotica shea butter (Butyrospermum parkii), galam butter (Butyrospermum parkii), Borneo butter or fat or tengkawang tallow (Shorea stenoptera), shorea butter, illipe butter, madhuca butter or Bassia madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter and sunflower butter.

An example of a preferred butter is shea butter.

In a known manner, shea butter is extracted from the fruit (also called “kernels” or “nuts”) of the Butyrospermum parkii tree. Each fruit contains between 45% and 55% fatty substance, which is extracted and generally refined.

According to a preferred embodiment, the fatty substances other than fatty acids that are useful according to the invention are chosen from solid fatty substances, preferably from solid fatty alcohols.

When they are present, the total content of the fatty substance(s) other than fatty acids preferably ranges from 5% to 35% by weight, more preferentially from 8% to 30% by weight and better still from 10% to 25% by weight, relative to the total weight of the composition (A).

In a particular embodiment, the composition (A) comprises one or more solid fatty substance(s) other than fatty acids, the total content of the solid fatty substance(s) other than fatty acids preferably ranging from 5% to 35% by weight, more preferentially from 8% to 30% by weight and better still from 10% to 25% by weight, relative to the total weight of the composition (A).

In another particular embodiment, the composition (A) comprises one or more liquid fatty substance(s) other than fatty acids, the total content of the liquid fatty substance(s) other than fatty acids preferably ranging from 0.5% to 15% by weight, more preferentially from 1% to 10% by weight and better still from 2% to 5% by weight, relative to the total weight of the composition (A).

According to a preferred embodiment, the composition (A) comprises one or more solid fatty substance(s) other than fatty acids, preferably chosen from solid fatty alcohols and one or more liquid fatty substance(s) other than fatty acids, preferably chosen from liquid fatty alcohols.

### Anionic Surfactants

The composition (A) may also comprise one or more anionic surfactants.

Preferably, the composition (A) comprises one or more anionic surfactants.

The term “anionic surfactant” means a surfactant including, as ionic or ionizable groups, only anionic groups. These anionic groups are preferably chosen from the following groups: CO₂H, CO₂⁻, SO₃H, SO₃⁻, OSO₂H, OSO₃⁻, H₂PO₃, HPO₃⁻, PO₃²⁻, H₂PO₂, HPO₂⁻, PO₂²⁻, POH and PO⁻.

As examples of anionic surfactants that may be used in the composition according to the invention, mention may be made of alkyl sulfates, alkyl ether sulfates, alkylamido ether sulfates, alkylaryl polyether sulfates, monoglyceride sulfates, alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates, acyl glutamates, alkyl sulfosuccinamates, acyl isethionates and N—(C1-C4)alkyl N-acyl taurates, salts of alkyl monoesters of polyglycoside-polycarboxylic acids, acyl lactylates, salts of D-galactoside uronic acids, salts of alkyl ether carboxylic acids, salts of alkylaryl ether carboxylic acids, salts of alkylamido ether carboxylic acids; fatty acid salts, and the corresponding non-salified forms of all these compounds; the alkyl and acyl groups of all these compounds (unless specified otherwise) generally including from 6 to 24 carbon atoms and the aryl group generally denoting a phenyl group.

These compounds may be oxyethylenated and then preferably include from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, they may be chosen from alkali metal salts such as the sodium or potassium salt and preferably the sodium salt, ammonium salts, amine salts and in particular amino alcohol salts or alkaline-earth metal salts such as the magnesium salt.

Examples of amino alcohol salts that may notably be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

The anionic surfactants that may be present may be mild anionic surfactants, i.e. anionic surfactants not bearing a sulfate function.

As regards the mild anionic surfactants, mention may be made in particular of the following compounds and salts thereof, and also mixtures thereof: polyoxyalkylenated alkyl ether carboxylic acids, polyoxyalkylenated alkylaryl ether carboxylic acids, polyoxyalkylenated alkylamido ether carboxylic acids, in particular those including 2 to 50 ethylene oxide groups, alkyl D-galactoside uronic acids, acyl sarcosinates, acyl glutamates and alkylpolyglycoside carboxylic esters.

Use may be made most particularly of polyoxyalkylenated alkyl ether carboxylic acids, for instance lauryl ether carboxylic acid (4.5 OE) sold, for example, under the name Akypo RLM 45 CA from Kao.

The anionic surfactants that may be present may be carboxylic acids comprising at least 8 carbon atoms, also known as fatty acids, optionally in salified form.

For the purposes of the present invention, the term “fatty acid” means an acid comprising at least one linear or branched, saturated or unsaturated hydrocarbon chain, such as an alkyl or alkenyl chain, including at least 6 carbon atoms, preferably from 8 to 24 carbon atoms, and better still from 10 to 22 carbon atoms.

The carboxylic acids comprising at least 6 carbon atoms (or fatty acids) according to the invention are neither (poly)oxyalkylenated, nor (poly)glycerolated; in particular, they are neither (poly)oxyethylenated, nor (poly)oxypropylenated.

They preferably have the structure R—COOH wherein R denotes a linear or branched C₇-C₂₉, preferably C₉-C₂₃ and better still C₉-C₁₇ alkyl or alkenyl group.

Preferably, the fatty acid according to the invention is chosen from linear fatty acids, better still from unsaturated linear C₁₀-C₂₂ and notably C₁₀-C₁₈ fatty acids (R is a linear C₉-C₂₃ or even C₉-C₁₇ alkenyl).

Mention may notably be made of oleic, linoleic, linolenic and undecylenic acids, and mixtures thereof. Preferably, oleic acid will be used.

Among the anionic surfactants mentioned above, fatty acids are preferably used.

Preferably, when the composition (A) comprises one or more anionic surfactants, the total content of anionic surfactant(s) in the composition preferably ranges from 0.01% to 15% by weight, more preferentially from 0.1% to 10% by weight, better still from 0.5% to 8% by weight, even better still from 1% to 6% by weight, relative to the total weight of the composition (A).

Preferably, when the composition (A) comprises one or more anionic surfactants chosen from fatty acids, the total content of fatty acids in the composition preferably ranges from 0.01% to 15% by weight, more preferentially from 0.1% to 10% by weight, better still from 0.5% to 8% by weight, even better still from 1% to 6% by weight, relative to the total weight of the composition (A).

### Sequestrants

The composition (A) may also comprise at least one sequestrant (or chelating agent).

Preferably, the composition (A) comprises one or more sequestrant(s).

The definition of a “sequestrant” (or “chelating agent”) is well known to those skilled in the art and refers to a compound or a mixture of compounds that are capable of forming a chelate with a metal ion. A chelate is an inorganic complex in which a compound (the sequestrant or chelating agent) is coordinated to a metal ion, i.e. it forms one or more bonds with the metal ion (formation of a ring including the metal ion).

A sequestrant (or chelating agent) generally comprises at least two electron-donating atoms which enable the formation of bonds with the metal ion.

Within the context of the present invention, the sequestrant(s) may be chosen from carboxylic acids, preferably aminocarboxylic acids, phosphonic acids, preferably aminophosphonic acids, polyphosphoric acids, preferably linear polyphosphoric acids, salts thereof, and derivatives thereof.

The salts are in particular alkali metal, alkaline-earth metal, ammonium and substituted ammonium salts.

The following compounds may be mentioned as examples of chelating agents based on carboxylic acids: diethylenetriaminepentaacetic acid (DTPA), ethylenediaminedisuccinic acid (EDDS) and trisodium ethylenediamine disuccinate such as Octaquest E30 from Octel, ethylenediaminetetraacetic acid (EDTA) and salts thereof such as disodium EDTA, tetrasodium EDTA, ethylenediamine-N,N′-diglutaric acid (EDDG), glycinamide-N,N′-disuccinic acid (GADS), 2-hydroxypropylenediamine-N,N′-disuccinic acid (HPDDS), ethylenediamine-N,N′-bis(ortho-hydroxyphenylacetic acid) (EDDHA), N,N′-bis(2-hydroxybenzyl)ethylenediamine-N,N′-diacetic acid (HBED), nitrilotriacetic acid (NTA), methylglycinediacetic acid (MGDA), N-2-hydroxyethyl-N,N-diacetic acid and glyceryliminodiacetic acid (as described in documents EP-A-317 542 and EP-A-399 133), iminodiacetic acid-N-2-hydroxypropylsulfonic acid and aspartic acid-N-carboxymethyl-N-2-hydroxypropyl-3-sulfonic acid (as described in EP-A-516 102), beta-alanine-N,N′-diacetic acid, aspartic acid-N,N′-diacetic acid, and aspartic acid-N-monoacetic acid (described in EP-A-509 382), chelating agents based on iminodisuccinic acid (IDSA) (as described in EP-A-509 382), ethanoldiglycine acid, phosphonobutane tricarboxylic acid such as the compound sold by Bayer under the reference Bayhibit AM, N,N-dicarboxymethylglutamic acid and salts thereof such as tetrasodium glutamate diacetate (GLDA) such as Dissolvine GL38 or 45S from Akzo Nobel.

The following compounds may be mentioned as examples of chelating agents based on mono- or polyphosphonic acid: diethylenetriaminepenta(methylene phosphonic acid) (DTPMP), ethane-1-hydroxy-1,1,2-triphosphonic acid (E1HTP), ethane-2-hydroxy-1,1,2-triphosphonic acid (E2HTP), ethane-1-hydroxy-1,1-triphosphonic acid (EHDP), ethane-1,1,2-triphosphonic acid (ETP), ethylenediaminetetramethylene phosphonic acid (EDTMP), hydroxyethane-1,1-diphosphonic acid (HEDP, or etidronic acid), and salts such as disodium etidronate, tetrasodium etidronate.

The following compounds may be mentioned as examples of chelating agents based on polyphosphoric acid: sodium tripolyphosphate (STP), tetrasodium diphosphate, hexametaphosphoric acid, sodium metaphosphate, phytic acid.

According to one embodiment, the sequestrant(s) that are useful according to the invention are phosphorus-based sequestrants, i.e. sequestrants which comprise one or more phosphorus atoms, preferably at least two phosphorus atoms.

The phosphorus-based sequestrant(s) used in the composition according to the invention are preferably chosen from:
inorganic phosphorus-based derivatives preferably chosen from alkali metal or alkaline-earth metal, preferably alkali metal, phosphates and pyrophosphates, such as sodium pyrophosphate, potassium pyrophosphate, sodium pyrophosphate decahydrate; and alkali metal or alkaline-earth metal, preferably alkali metal, polyphosphates, such as sodium hexametaphosphate, sodium polyphosphate, sodium tripolyphosphate, sodium trimetaphosphate; which are optionally hydrated, and mixtures thereof;
organic phosphorus-based derivatives, such as organic (poly)phosphates and (poly)phosphonates, such as etidronic acid and/or alkali metal or alkaline-earth metal salts thereof, for instance tetrasodium etidronate, disodium etidronate, and mixtures thereof.

Preferably, the phosphorus-based sequestrant(s) is/are chosen from linear or cyclic compounds comprising at least two phosphorus atoms bonded together covalently via at least one linker L comprising at least one oxygen atom and/or at least one carbon atom.

The phosphorus-based sequestrant(s) may be chosen from inorganic phosphorus-based derivatives, preferably comprising at least two phosphorus atoms. More preferentially, the phosphorus-based sequestrant(s) is/are chosen from alkali metal or alkaline-earth metal pyrophosphates, better still from alkali metal pyrophosphates, in particular sodium pyrophosphate (also known as tetrasodium pyrophosphate).

The phosphorus-based sequestrant(s) may be chosen from organic phosphorus-based derivatives, preferably comprising at least two phosphorus atoms. More preferentially, the phosphorus-based sequestrant(s) is/are chosen from etidronic acid (also known as 1-hydroxyethane-1,1-diphosphonic acid) and/or alkali metal or alkaline-earth metal, preferably alkali metal, salts thereof, for instance tetrasodium etidronate and disodium etidronate.

Thus, preferably, the phosphorus-based sequestrant(s) are chosen from alkali metal pyrophosphates, etidronic acid and/or alkali metal salts thereof, and a mixture of these compounds.

Particularly preferably, the phosphorus-based sequestrant(s) are chosen from tetrasodium etidronate, disodium etidronate, etidronic acid, tetrasodium pyrophosphate, and a mixture of these compounds.

According to the present invention, the sequestrants are preferably chosen from diethylenetriaminepentaacetic acid (DTPA) and salts thereof, diethylenediaminetetraacetic acid (EDTA) and salts thereof, ethylenediaminedisuccinic acid (EDDS) and salts thereof, etidronic acid and salts thereof, N,N-dicarboxymethylglutamic acid and salts thereof (GLDA), and mixtures thereof.

More preferentially, the sequestrant(s) are chosen from N,N-dicarboxymethylglutamic acid and salts thereof (GLDA), and mixtures thereof.

Among the salts of these compounds, the alkali metal salts and notably the sodium or potassium salts are preferred.

When the composition (A) comprises one or more sequestrants, the total content of the sequestrant(s) preferably ranges from 0.001% to 15% by weight, more preferentially from 0.005% to 10% by weight, better still from 0.01% to 8% by weight, even better still from 0.05% to 5% by weight, relative to the total weight of the composition (A).

According to a particular embodiment, the composition (A) comprises:
at least one fatty substance other than fatty acids;
at least one alkyl (poly)glycoside;
at least one polysaccharide;
at least one alkaline agent and
at least one dye chosen from direct dyes, oxidation dyes and mixtures thereof, preferably at least one dye chosen from oxidation dyes and mixtures thereof.

According to a particular embodiment, the composition (A) according to the invention comprises:
at least one fatty substance other than fatty acids, preferably at least one liquid fatty substance and at least one solid fatty substance;
au moins un alkyl (poly)glycoside;
at least one polysaccharide, preferably chosen from anionic polysaccharides;
at least one alkaline agent;
at least one dye chosen from direct dyes, oxidation dyes and mixtures thereof, preferably at least one dye chosen from oxidation dyes and mixtures thereof;
at least one sequestrant, preferably chosen from diethylenetriaminepentaacetic acid (DTPA) and salts thereof, diethylenediaminetetraacetic acid (EDTA) and salts thereof, ethylenediaminedisuccinic acid (EDDS) and salts thereof, etidronic acid and salts thereof, N,N-dicarboxymethylglutamic acid and salts thereof (GLDA), and mixtures thereof.

### Solvents

The composition (A) may also comprise at least one organic solvent.

Preferably, the composition (A) comprises at least one organic solvent.

Examples of organic solvents that may be mentioned include linear or branched C₂ to C₄ alkanols, such as ethanol and isopropanol; polyols and polyol ethers, for instance 2-butoxyethanol, propylene glycol, glycerol, 1,3-propanediol, dipropylene glycol, propylene glycol monomethyl ether, diethylene glycol monomethyl ether and monoethyl ether, and also aromatic alcohols or ethers, for instance benzyl alcohol or phenoxyethanol, and mixtures thereof.

Preferably, the organic solvent(s) is (are) chosen from polyols, more preferentially glycerol, 1,3-propanediol and mixtures thereof.

The organic solvent(s) may be present in a total amount ranging from 0.01% to 30% by weight, preferably ranging from 2% to 25% by weight, relative to the total weight of the composition (A).

In addition, preferably, the composition (A) is an aqueous composition. The composition (A) preferably comprises water in an amount of greater than or equal to 5% by weight, preferably greater than or equal to 10% by weight, and better still greater than or equal to 15% by weight, relative to the total weight of the composition (A).

Preferably, the composition (A) comprises water in an amount ranging from 5% to 90% by weight, preferably from 10% to 80% by weight, and better still from 15% to 75% by weight, relative to the total weight of the composition (A).

### Additives

The composition (A) may optionally comprise one or more additives, other than the compounds of the invention and among which mention may be made of cationic, anionic, nonionic or amphoteric polymers or mixtures thereof, other than those mentioned previously, antidandruff agents, anti-seborrhoeic agents, agents for preventing hair loss and/or for promoting hair regrowth, vitamins and provitamins including panthenol, sunscreens, mineral or organic pigments, plasticizers, solubilizers, opacifiers or nacreous agents, antioxidants, hydroxy acids, fragrances, and preserving agents.

Needless to say, those skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight, relative to the total weight of the composition (A).

Preferably, the composition (A) does not comprise any chemical oxidizing agents.

### 2. Oxidizing Composition (B)

The oxidizing composition (B) comprises one or more chemical oxidizing agents.

According to the invention, the term “chemical oxidizing agent” means an oxidizing agent other than atmospheric oxygen.

More particularly, the chemical oxidizing agent(s) are chosen from hydrogen peroxide, urea peroxide, alkali metal bromates or ferricyanides, peroxygenated salts, for instance persulfates, perborates, peracids and precursors thereof and percarbonates of alkali metals or alkaline-earth metals, and mixtures thereof. The oxidizing agent is preferably chosen from hydrogen peroxide.

The oxidizing composition (B) is preferably an aqueous composition. In particular, it comprises more than 5% by weight of water, preferably more than 10% by weight of water and even more advantageously more than 20% by weight of water.

Preferably, the oxidizing composition (B) comprises water in an amount ranging from 5% to 95% by weight, preferably from 10% to 92% by weight, and better still from 15% to 90% by weight, relative to the total weight of the oxidizing composition (B).

It may also comprise one or more organic solvents chosen from those listed previously; these solvents more particularly representing, when they are present, from 1% to 40% by weight and preferably from 5% to 30% by weight, relative to the weight of the oxidizing composition.

The oxidizing composition (B) also preferably comprises one or more acidifying agents. Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

The oxidizing composition (B) may also comprise fatty substances such as those described above, preferably chosen from fatty alcohols, liquid hydrocarbons comprising more than 16 carbon atoms and mixtures thereof, surfactants and polymers.

Usually, the pH of the oxidizing composition, when it is aqueous, is less than 7.

Preferably, the oxidizing composition (B) comprises hydrogen peroxide as oxidizing agent, the concentration of which ranges, more particularly, from 0.1% to 50%, more particularly between 0.5% and 20% and even more preferentially between 1% and 15% by weight, relative to the weight of the oxidizing composition (B).

The composition (A) and the composition (B) are preferably mixed in a composition (A)/composition (B) weight ratio ranging from 0.5 to 2, preferentially from 0.5 to 1.5 and better still from 0.5 to 1.

The ready-to-use composition (C) resulting from the mixing of the dyeing and/or lightening composition (A) and of the oxidizing composition (B) preferably has a viscosity of greater than or equal to 3 Pa·s, preferably greater than or equal to 3.2 Pa·s, better still greater than or equal to 3.5 Pa·s at a temperature of 25° C., at atmospheric pressure and at a shear rate of 1 s-1 (measurable, for example, with a Haake RS600 rheometer). Preferably, the ready-to-use composition (C) has a viscosity ranging from 3 to 500 Pa·s, more preferentially ranging from 3.2 to 300 Pa·s, and even more preferentially ranging from 3.5 to 200 Pa·s at a temperature of 25° C., at atmospheric pressure and at a shear rate of 1 s-1 (measurable, for example, with a Haake RS600 rheometer).

Preferably, the ready-to-use composition (C) resulting from the mixing of the dyeing and/or lightening composition (A) and of the oxidizing composition (B) has a threshold stress at 25° C. of greater than or equal to 10 Pa, preferably greater than or equal to 10.2 Pa, better still greater than or equal to 10.3 Pa. Preferably, the ready-to-use composition (C) has a threshold stress at 25° C. ranging from 10 to 300 Pa, preferably from 10.2 to 250

Pa and more preferably from 10.3 to 200 Pa.

The threshold stress is determined by scanning under stress at 25° C. An imposed-stress Thermo Haake RS600 rheometer with sandblasted cone-plate geometry is used. The temperature is regulated by a Peltier-effect plate and an anti-evaporation device (solvent trap filled with water for the measurements at 25° C.).

A logarithmic stress elevation from 0.5 to 500 Pa is performed over a period of 3 minutes. Two adjustment lines corresponding to the stationary regimes (solid and liquid behaviors) are plotted on the curve representing the strain as a function of the stress (logarithmic coordinates). The intersection of these two lines gives the value of the stress threshold.

### 3. Pot

The pot used in the process according to the invention for receiving the mixture of the compositions (A) and (B) and obtaining the ready-to-use composition (C) has a base with an inside area S, the base forming the bottom of the pot.

The pot serves both to separately contain the compositions (A) and (B) to be mixed and to mix them.

The volume V occupied by the ready-to-use composition (C) resulting from the mixing of the composition (A) and of the oxidizing composition (B) is preferably between 70 and 150 ml, and more preferentially between 95 and 125 ml.

The ratio V/S may be between 8 and 30 mm, preferably between 13 and 20 mm, more preferably between 15.0 and 18.5 mm. The ratio V/S corresponds substantially to the height of the mixture.

The base of the pot has an internal diameter D.

The pot is preferably of circular cross section, with an internal diameter D of the base preferably of between 80 and 105 mm, better still between 90 and 95 mm. The term “internal diameter” should be understood as meaning, when the cross section is non-circular, the equivalent diameter of an imaginary circular cross section of the same area.

A pot of which the base is not circular, in particular a pot with a substantially rectangular base, may also be used. The rectangular base has, for example, dimensions of 90 mm×70 mm, which is substantially equivalent to a circular base with a diameter of 90 mm.

The pot preferably has an internal height not exceeding 125 mm, better still not exceeding 110 mm. The internal height can in particular be between 50 and 110 mm.

The dimensions of the pot make it possible, on the one hand, to limit the risk of overflowing and guarantee, on the other hand, a sufficient level of product in the pot to facilitate its homogenization and access with the hand to the bottom of the pot for taking a sample of the product.

Moreover, such a pot exhibits an internal volume making it possible to house all the constituents of the assembly with a satisfactory degree of filling of the pot, which prevents having to provide specific means for wedging the various constituents.

The pot contains in particular the compositions to be mixed in order to obtain the ready-to-use composition (C).

The compositions (A) and (B) to be mixed are packaged, separately, in a container which may be a bag, a tube or a bottle. Preferably, each composition (A) and (B) is packaged in a bag.

Each composition (A) and (B) can be present in the pot in a volume ranging from 40 to 75 ml.

The pot may also contain other elements that are of use in the dyeing and/or lightening process of the invention.

It may for example contain gloves to protect the hands of the user during application.

It may optionally contain a spatula for stirring the mixture of the compositions (A) and (B).

It may also contain one or more other compositions to be applied after performing the dyeing and/or the lightening, for example a care composition or a shampoo.

It may also contain instructions for use.

The pot may comprise a lid arranged to be attached on top so as to enclose the various constituents initially contained in the pot.

The material of the pot can be of any cardboard suitable for containing the mixture for the period of time necessary for the application. The lid can also be made of cardboard. The spatula can be made of wood, of compressed palm or banana leaf, or also made of folded cardboard of the same nature as the pot, if the latter is made of cardboard.

The use of cardboard makes it possible to reduce the amount of plastic, and the pot can be a single-use pot, which prevents having to carry out thorough water-consuming cleaning.

A better understanding of the invention can be obtained on reading the detailed description which will follow of non-limiting implementational examples of the invention and on examining the appended drawing, wherein:

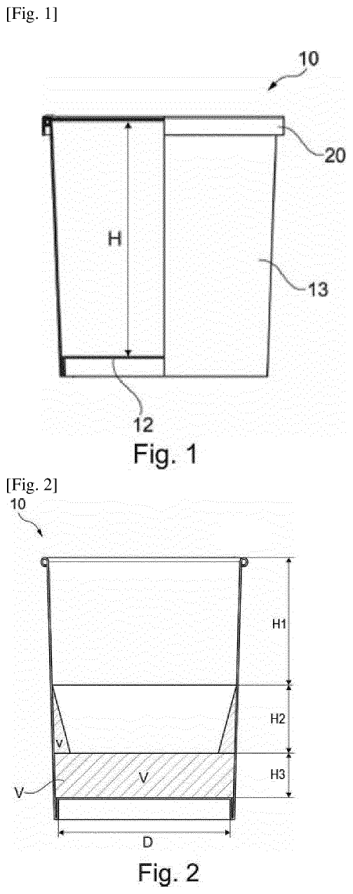

FIG. 1 represents the pot and the lid;

FIG. 2 represents in isolation, in longitudinal cross section, the pot containing the mixture of the compositions.

According to the particular embodiment illustrated in FIGS. 1 and 2, the pot 10 has a body 13 with a general frustoconical shape.

The pot 10 can comprise an added bottom 12, having a cross section in the shape of an inverted U, adhesively bonded by its periphery to the body 13.

The diameter D is that measured inside the pot at its base, that it is say at the wall of the added bottom.

The interior height H of the pot corresponds to the distance between the bottom 12 and the plane resting on the upper edge of the pot 10.

The internal diameter D of the base of the pot 10 is, for example, between 80 and 105 mm, i.e. an inside area S of the base approximately of between 50 and 87 cm².

The assembly comprises a lid 20 for closing the pot 10.

The pot is made of a cardboard material. The added bottom is also made of a cardboard material, as is the lid 20.

The compositions (A) and (B) form, after having been poured into the pot, a total volume V of ready-to-use composition (C).

This volume V occupies a height H3 in the pot 10, as illustrated in FIG. 2.

During the mixing of the compositions (A) and (B) by stirring using a spatula, a small volume v of mixture typically wets a height H2 of the wall of the pot above the level of the mixture at rest.

The remaining height H1 is such that the internal height H=H1+H2+H3 is between 50 and 110 mm.

For example, H2 is between 10 and 50 mm and H1 is between 32 and 60 mm.

Each composition (A) and (B) is packaged in a bag, which may be a flat bag.

The pot 10 initially contains the bags containing the compositions (A) and (B). It also contains a pair of gloves, a mixing spatula, and a third bag containing a hair care composition. It may also contain instructions for use.

### Process

According to the dyeing and/or lightening process of the invention, the user simultaneously or successively pours, in step a), a dyeing and/or lightening composition (A) as described above and an oxidizing composition (B) as described above into a pot as described above, having a base with an inside area S.

Preferably, the user pours the oxidizing composition (B) into the pot before the composition (A) in step a).

The user then mixes the two compositions (A) and (B) in the pot in order to obtain a ready-to-use composition (C) occupying a volume V in the pot, the ratio V/S being between 8 and 30 mm.

The step of mixing the compositions (A) and (B) is preferably carried out with a spatula. As a variant, it is possible to dispense with the spatula and to mix the two compositions directly with the hand.

The user then takes a sample of the composition (C) from the pot by hand and applies it to the hair by hand. These steps can be repeated between one and ten times to apply the required amount of composition (C) to the hair so that all of the hair of a head is covered with composition (C).

According to one preferred embodiment, the user takes a small amount of composition (C) by hand, preferably an amount corresponding to a knob, and applies it to the hair. Said user reproduces this step several times until all of the hair of the head is covered with composition (C).

According to one preferred embodiment, in step d), the user first applies the composition (C) to the roots of the hair of the head, preferably on either side of a line defined beforehand, preferably on the upper part of the head. The user can repeat this step by successively applying on either side of several lines formed on the upper part of the head, until all of the roots of the upper part of the head and the temples are covered with composition (C).

Preferably, after having applied the composition (C) to all the roots of the upper part of the head and the temples, the user attaches the lengths of hair of the upper part of the head, so as to free the back part of the head. If the hair is sufficiently long, the user preferably forms a bun with the hair of the upper part of the head. The user then applies the composition (C) to the roots of the back part of the head.

Preferably, the user spreads the composition (C) present on the roots over all the lengths of hair, by hand. When the user has attached the hair of the upper part of the head beforehand, he or she releases the hair, in particular the bun, before spreading the composition (C) present on the roots over all the lengths of hair, by hand.

After having applied the composition (C) to the hair, the user can optionally massage the hair with the hand.

After a leave-on time ranging from 1 min to 1 hour, preferably from 10 to 50 min, preferentially from 15 to 45 min, the user rinses the hair.

Preferably, when the compositions (A) and (B) are initially contained in containers, preferably bags, packaged in the pot, the user must first remove the containers from the pot before opening them to pour their content into the pot.

Preferably, when the pot packages other constituents, such as protective gloves, a mixing spatula, another container containing another composition such as a hair care product, instructions for use, etc., the user must of course remove them from the pot before pouring the compositions (A) and (B) into said pot.

The ready-to-use composition (C) can be applied by hand to dry or wet hair.

At the end of the treatment, after rinsing, the hair is optionally subjected to a step of washing g) the hair with a detergent composition (D), followed by a step of rinsing with water, optionally followed by a step of wringing out h) the hair.

A hair care composition (E) can optionally be applied after rinsing the hair, optionally followed by another rinsing step.

At the end of treatment, the hair can be dried or left to dry.

Preferably, the user covers their hands with protective gloves before taking a sample of the composition by hand in step c), preferably before the mixing step b), or preferably before, in step a), pouring the compositions (A) and (B) into the pot.

Preferably, the user's hands are covered with protective gloves at least up to the hair-rinsing step f).

The following examples serve to illustrate the invention without, however, being limiting in nature.

### EXAMPLES

In the examples that follow, all the amounts are given as mass percentages of active material (AM) relative to the total weight of the composition (unless otherwise mentioned).

### Compositions

The dyeing compositions A1 to A3 and the oxidizing composition B were prepared from the ingredients, the contents of which are indicated in the table below (as % of active material):

### Dye Compositions

TABLE 1

| | A1 | A2 | A3 |
|---|---|---|---|
| Xanthan gum | 0.2 | 0.2 | 0.2 |
| Oleic acid | 2.7 | 2.7 | 2.7 |
| Ethanolamine | 12.2 | 12.2 | 12.3 |
| Cetearyl alcohol | 16.3 | 16.3 | 16.3 |
| Toluene-2,5-diamine | — | 0.68 | — |
| Hydroxybenzomorpholine | — | 0.49 | 0.95 |
| 2,4-Diaminophenoxyethanol HCl | 0.016 | 0.028 | 0.12 |
| 6-Hydroxyindole | — | 0.069 | 0.1 |
| N,N-Bis(2-hydroxyethyl)-p-phenylenediamine | 0.058 | 0.044 | 0.13 |
| sulfate | | | |
| 2-Amino-3-hydroxypyridine | — | 0.009 | 0.04 |
| Resorcinol | 1.2 | — | — |
| m-Aminophenol | 0.09 | 0.216 | 0.53 |
| p-Phenylenediamine | 1 | — | — |
| Hydroxyethyl-3,4-methylenedioxyaniline HCl | — | 0.12 | — |
| 2-METHOXYMETHYL-P-PHENYLENEDIAMINE | — | — | 1.8 |
| 1,3-Propanediol | 5 | 5 | 5 |
| Ascorbic acid | 0.25 | 0.25 | 0.25 |
| Tetrasodium glutamate diacetate | 0.24 | 0.24 | 0.24 |
| Cetearyl glucoside | 0.5 | 0.5 | 0.5 |
| Glycerol | 5 | 5 | 5 |
| Oleyl alcohol | 2.7 | 2.7 | 2.7 |
| Sodium metabisulfite | 0.7 | 0.7 | 0.7 |
| Water | q.s. 100 | q.s. 100 | q.s. 100 |

### Oxidizing Composition

TABLE 2

| | B |
|---|---|
| Trideceth-2 carboxamide MEA | 0.85 |
| Tetrasodium etidronate | 0.06 |
| Sodium salicylate | 0.035 |
| Glycerol | 0.5 |
| Cetearyl alcohol | 2.28 |
| Ceteareth-25 | 0.57 |
| Phosphoric acid | qs pH = 2.2 ± 0.2 |
| Hydrogen peroxide | 6 |
| Tetrasodium pyrophosphate | 0.04 |
| Water | q.s. 100 |

A pot as described above and illustrated in FIGS. 1 and 2 contains a first bag containing 40 g of dyeing composition A1 and a second bag containing 60 g of oxidizing composition B. The pot also contains instructions for use, a pair of gloves, a wooden spatula and a third bag containing a hair care composition.

The pot has a base of circular cross section. The internal diameter D of the base is approximately equal to 92 mm. The internal height H of the pot is approximately equal to 108 mm.

The user removes from the pot all the elements initially contained therein.

He or she puts on the protective gloves before pouring the entire contents of the bag containing the oxidizing composition B and then that of the bag containing the dyeing composition A1 into the pot.

This is followed by mixing of the two compositions A1 and B using the spatula, so as to obtain a ready-to-use composition C occupying a volume of approximately 100 ml in the pot. The mixing is easy.

The user then takes a knob of composition (C) from the pot by hand and applies it to their dry hair by hand.

These steps of taking a sample and applying it by hand are repeated so as to apply the required amount of composition (C) to the hair.

It is easy to take a sample of the dyeing mixture by hand right down to the bottom of the pot, without the risk of touching the inner lateral wall of the pot with the wrist. The application to the hair by hand is easy and allows good localization of the dyeing mixture at the desired places, without running.

The user massages their hair with the hand so as to distribute the composition over the entire head of hair.

After a leave-on time of 30 min, the user rinses their hair with water. Their hair is then left to dry.

A powerful dyeing of the hair is obtained.

According to two other examples of implementation, the pot contains a bag containing the dyeing composition A2 instead of the composition A1, or the composition A3. The same process as that described in the first example is implemented.

The compositions A2 and A3 also result in powerful colorings of the hair.

1-33. (canceled)

34. A process for dyeing and/or lightening keratin fibers, comprising:
a) simultaneously or successively pouring (i) a dyeing and/or lightening composition (A) comprising at least one alkaline agent, at least one dyeing agent, or a mixture thereof, and (ii) an oxidizing composition (B) comprising at least one chemical oxidizing agent into a pot having a base with an inside area S;
b) mixing the two compositions (A) and (B) in the pot to obtain a composition (C) occupying a volume V in the pot, the ratio V/S ranging from 8 mm to 30 mm;
c) taking a sample of the composition (C) from the pot by hand;
d) applying the composition (C) to the keratin fibers by hand;
e) optionally, hand-massaging the keratin fibers;
f) after a leave-on time ranging from about 1 min to about 1 hour, rinsing the keratin fibers.

35. The process of claim 34, wherein the composition (A) further comprises at least one alkyl (poly)glycoside.

36. The process of claim 34, wherein the at least one alkyl (poly)glycoside is chosen from compounds of formula R₁O—(R₂O)t-(G)v, wherein:
R₁ represents a linear or branched alkyl or alkenyl radical comprising 6 to 24 carbon atoms, or an alkylphenyl radical of which the linear or branched alkyl radical comprises 6 to 24 carbon atoms,
R₂ represents an alkylene radical comprising 2 to 4 carbon atoms,
G represents a sugar unit comprising 5 to 6 carbon atoms,
t denotes an integer ranging from 0 to 10,
v represents a degree of polymerization and denotes an integer ranging from 1 to 15.

37. The process of claim 34, wherein the total amount of alkyl (poly)glycoside(s) in the composition (A) ranges from 0.01% to 20% by weight, relative to the total weight of the composition (A).

38. The process of claim 34, wherein composition (A) further comprises at least one fatty substance other than a fatty acid, the at least one fatty substance chosen from a liquid fatty substance, a solid fatty substance or mixtures thereof.

39. The process of claim 38, wherein the total amount of the at least one fatty substance other than a fatty acid in composition (A) ranges from 5% to 35% by weight, relative to the total weight of composition (A).

40. The process of claim 34, wherein composition (A) further comprises at least one polysaccharide.

41. The process of claim 40, wherein the total amount of the at least one polysaccharide in composition (A) ranges from 0.01% to 10% by weight, relative to the total weight of composition (A).

42. The process of claim 34, wherein composition (A) comprises at least one dyeing agent chosen from oxidation dyes.

43. The process of claim 34, wherein composition (A) comprises at least one oxidation dye chosen from 6-hydroxybenzomorpholine, the addition salts thereof, the solvates thereof and/or the solvates of the salts thereof; hydroxyethyl-3,4-methylenedioxyaniline, the addition salts thereof, the solvates thereof and/or the solvates of the salts thereof; 2-amino-5-ethylphenol, the addition salts thereof, the solvates thereof and/or the solvates of the salts thereof; or mixtures thereof.

44. The process of claim 34, wherein composition (A) comprises at least one alkaline agent chosen from alkanolamines; aqueous ammonia; carbonates or bicarbonates; alkali metal or alkaline-earth metal silicates or metasilicates; basic amino acids; or mixtures thereof.

45. The process of claim 34, wherein the total amount of the at least one alkaline agent ranges from 0.1% to 40% by weight, relative to the total weight of composition (A).

46. The process of claim 34, wherein in step a), the oxidizing composition (B) is poured into the pot before the dyeing and/or lightening composition (A).

47. The process of claim 34, wherein composition (C) which results from mixing composition (A) with composition (B) has a viscosity of greater than or equal to 3 Pa·s and a threshold stress of greater than or equal to 10 Pa.

48. The process of claim 34, wherein steps c) and d) are repeated between one and ten times before optional step e) or before step f) so that all of the hair of a head is covered with composition (C).

49. The process of claim 34, wherein the pot has an internal height (H) equal to or less than 125 mm, a circular cross section, and a base with an internal diameter (D) ranging from 80 nm to 105 nm.

50. The process of claim 34, wherein the volume V occupied by composition (C) ranges from 70 mL to 150 mL, and the ratio of V/S ranges from 13 nm to 20 nm.

51. The process of claim 34, wherein, before step a), composition (A) and composition (B) are each contained respectively in a container, each container being packaged in the pot, the process comprising, before step a), a step of removing the containers from the pot.

52. The process of claim 34, further comprising, after step f), a step g) washing the hair with a detergent composition, optionally followed by a step h) wringing out the hair.

53. The process of claim 34, further comprising, after step f), or after step g) or after step h) if they are present, a step i) applying a hair care composition, optionally followed by a j) rinsing step.
